(19) United States
(12) Patent Application Publication
(10) Pub. No.: US 2006/0029962 A1
(43) Pub. Date: Feb. 9, 2006

(54) Assays for TERT promoter modulatory agents

(76) Inventors: William H. Andrews, Reno, NV (US); Bryant Villeponteau, San Diego, CA (US); Laura A. Briggs, Reno, NV (US); Trenton D. Johnson, Reno, NV (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/198,933

(22) Filed: Aug. 5, 2005

Related U.S. Application Data
(60) Provisional application No. 60/618,484, filed on Oct. 12, 2004.
(60) Provisional application No. 60/599,351, filed on Aug. 6, 2004.

(51) Int. Cl. C12Q 1/68

(52) U.S. Cl. 435/6

(57) ABSTRACT

Methods and compositions for assaying an agent for TERT promoter modulatory activity are provided. In the subject methods, an agent is contacted with an expression system that includes a TERT promoter nucleic acid operably linked to a heterologous reporter nucleic acid. A feature of the expression system is that it is integrated into a carrier nucleic acid, e.g., a chromosome, in a manner such that it is known to be inactive under wild-type conditions but not positionally silenced. Also provided are compositions, systems and kits thereof, as well as devices, that find use in practicing the subject methods. The subject invention finds use in assaying agents for TERT promoter modulatory activity, such as in a high throughput format.

### CROSS-REFERENCE TO RELATED APPLICATIONS

Pursuant to 35 U.S.C. § 119 (e), this application claims priority to the filing date of U.S. Provisional Patent Application Ser. No. 60/618,484 filed Oct. 12, 2004 and to the filing date of U.S. Provisional Patent Application Ser. No. 60/599,351 filed Aug. 6, 2004; the disclosures of which applications are herein incorporated by reference.

### INTRODUCTION

1. Background of the Invention

Telomeres, which define the ends of chromosomes, consist of short, tandemly repeated DNA sequences loosely conserved in eukaryotes. For example, human telomeres consist of many kilobases of (TTAGGG)n together with various associated proteins. Small amounts of these terminal sequences or telomeric DNA are lost from the tips of the chromosomes during S phase because of incomplete DNA replication. Many human cells progressively lose terminal sequence with cell division, a loss that correlates with the apparent absence of telomerase in these cells. The resulting telomeric shortening has been demonstrated to limit cellular lifespan.

Telomerase is a ribonucleoprotein that synthesizes telomeric DNA. In general, telomerase is made up of two components: (1) an essential structural RNA (TR or TER) (where the human component is referred to in the art as hTR or hTER); and (2) a catalytic protein (telomerase reverse transcriptase or TERT) (where the human component is referred to in the art as hTERT). Telomerase works by recognizing the 3′ end of DNA, e.g., telomeres, and adding multiple telomeric repeats to its 3′ end with the catalytic protein component, e.g., hTERT, which has polymerase activity, and hTER which serves as the template for nucleotide incorporation. Of these two components of the telomerase enzyme, both the catalytic protein component and the RNA template component are activity-limiting components.

Because of its role in cellular senescence and immortalization, there is much interest in the development of protocols and compositions for regulating telomerase activity.

2. Literature of Interest

U.S. Pat. Nos. 5,972,605; 6,610,839 and 6,664,046 and published United States Application 2004/0072787; as well as WO 02/070668; WO 03/016474; WO 03/000916; WO 02/101010; WO 02/090571; WO 02/090570; WO 02/072787; WO 02/070668; WO 02/16658; WO 02/16657; and the references cited therein.

### SUMMARY OF THE INVENTION

Methods and compositions for assaying an agent for TERT promoter modulatory activity are provided. In the subject methods, an agent is contacted with an expression system that includes a TERT promoter nucleic acid operably linked to a heterologous reporter nucleic acid. A feature of the expression system is that it is integrated into a carrier nucleic acid, e.g., a chromosome, in a manner such that it is known to be inactive under wild-type conditions but not positionally silenced. Also provided are compositions, systems and kits thereof, as well as devices, that find use in practicing the subject methods. The subject invention finds use in assaying agents for TERT promoter modulatory activity, such as in a high throughput format.

### BRIEF DESCRIPTION OF THE FIGURES

FIG. 1 shows the hTERT promoter sequence including Site C.

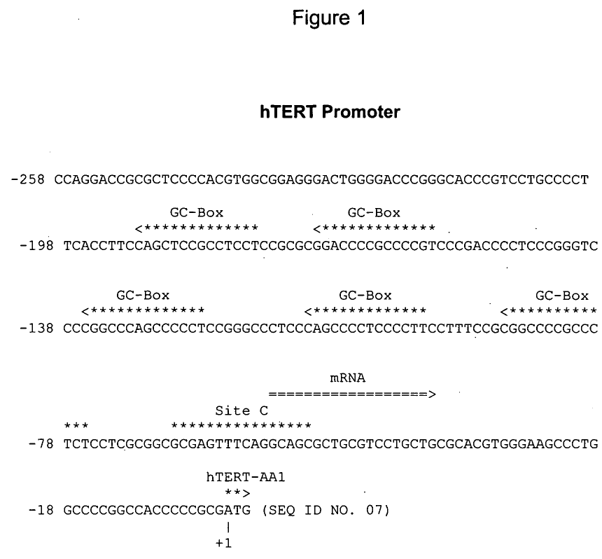

FIG. 2 provides a schematic view of a representative high-throughput screening device according to an embodiment of the invention (see Example I).

FIG. 3 is a diagram of an exemplary embodiment of depicting generating test and reporter cell lines according to the subject invention (see Example ll).

### DEFINITIONS

As used herein, the terms “allele” or “allelic sequence” refer to an alternative form of a nucleic acid sequence (i.e., a nucleic acid corresponding to a TERT promoter, particularly, an hTERT promoter). Alleles result from mutations (i.e., changes in the nucleic acid sequence), and can produce differently regulated mRNAs. Common mutational changes that give rise to alleles are generally ascribed to natural deletions, additions, or substitutions of nucleotides. Each of these types of changes may occur alone, in combination with the others, or one or more times within a given gene, chromosome or other cellular nucleic acid. Thus, the term “TERT promoter” includes allelic forms of TERT promoter sequences, i.e., TERT cis-acting transcriptional control elements, including, e.g., the exemplary human and mouse sequences described herein.

The term “amplifying” as used herein incorporates its common usage and refers to the use of any suitable amplification methodology for generating or detecting recombinant or naturally expressed nucleic acid, as described in detail, below. For example, the invention provides methods and reagents (e.g., specific oligonucleotide PCR primer pairs) for amplifying (e.g., by PCR) sequences expressed under the control of a TERT promoter in vivo or in vitro.

As used herein, the term “TERT promoter” includes any TERT genomic sequences capable of driving transcription in a telomerase activity positive cell. Thus, TERT promoters of the invention include without limitation cis-acting transcriptional control elements and regulatory sequences that are involved in regulating or modulating the timing and/or rate of transcription of a TERT gene. For example, the TERT promoter of the invention comprises cis-acting transcriptional control elements, including enhancers, promoters, transcription terminators, origins of replication, chromosomal integration sequences, 5′ and 3′ untranslated regions, exons and introns, which are involved in transcriptional regulation. These cis-acting sequences typically interact with proteins or other biomolecules to carry out (turn on/off, regulate, modulate, etc.) transcription.

In alternative embodiments, the TERT promoter sequence comprises TERT sequences 5′ (upstream) of the translational start site (ATG). For example, in one embodiment, the hTERT promoter comprises residues 44 to 13545 of SEQ ID NO:05. Other embodiments include sequences starting within about one to 5 nucleotides of a translation start codon (for example in SEQ ID NO:05 or SEQ ID NO:06) and ending at about 50, 100, 150, 200, 250, 500, 1000, 2500 or 13500 nucleotides upstream of the translation start codon. Such embodiments can optionally include other regulatory sequences, such as, exon and/or intron sequences. hTERT promoters of the invention also include sequences substantially identical (as defined herein) to an exemplary hTERT promoter sequence of the invention, having the sequence set forth by SEQ ID NO:05. Similarly, mTERT promoters of the invention also include sequences substantially identical to an exemplary mTERT promoter sequence of the invention, having the sequence set forth by SEQ ID NO:06.

The term “heterologous” when used with reference to portions of a nucleic acid, indicates that the nucleic acid comprises two or more subsequences which are not found in the same relationship to each other in nature. For instance, the nucleic acid is typically recombinantly produced, having two or more sequences from unrelated genes arranged in a manner not found in nature; e.g., a promoter sequence of the invention operably linked to a polypeptide coding sequence that, when operably linked, does not reform the naturally occurring TERT gene. For example, the invention provides recombinant constructs (expression cassettes, vectors, viruses, and the like) comprising various combinations of promoters of the invention, or subsequences thereof, and heterologous coding sequences, many examples of which are described in detail below.

As used herein, “isolated,” when referring to a molecule or composition, such as, e.g., an hTERT promoter sequence, means that the molecule or composition is separated from at least one other compound, such as a protein, DNA, RNA, or other contaminants with which it is associated in vivo or in its naturally occurring state. Thus, a nucleic acid sequence is considered isolated when it has been isolated from any other component with which it is naturally associated. An isolated composition can, however, also be substantially pure. An isolated composition can be in a homogeneous state. It can be in a dry or an aqueous solution. Purity and homogeneity can be determined, e.g., using analytical chemistry techniques such as, e.g., polyacrylamide gel electrophoresis (PAGE), agarose gel electrophoresis or high pressure liquid chromatography (HPLC).

As used herein, the terms “nucleic acid” and “polynucleotide” are used interchangeably, and include oligonucleotides (i.e., short polynucleotides). They also refer to synthetic and/or non-naturally occurring nucleic acids (i.e., comprising nucleic acid analogues or modified backbone residues or linkages). The terms also refer to deoxyribonucleotide or ribonucleotide oligonucleotides in either single-or double-stranded form. The terms encompass nucleic acids containing known analogues of natural nucleotides. The term also encompasses nucleic acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene (methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NTYAS 1992);. Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described in WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (Strauss-Soukup (1997) Biochemistry 36:8692-8698), and benzyl-phosphonate linkages (Samstag (1996) Antisense Nucleic Acid Drug Dev 6:153-156).

As used herein, the term “operably linked” refers to a functional relationship between two or more nucleic acid (e.g., DNA) segments. Typically, it refers to the functional relationship of a transcriptional regulatory sequence to a transcribed sequence. For example, a TERT promoter sequence of the invention, including any combination of cis-acting transcriptional control elements, is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” also encompass the ligation of nucleic acids having coding or promoter sequences from different sources into an expression cassette or vector for expression of, e.g., a fusion protein; or, inducible, constitutive expression of a protein (i.e., a TERT promoter of the invention operably linked to a heterologous nucleotide, such as a polypeptide coding sequence).

As used herein, the “sequence” of a gene (unless.specifically stated otherwise) or nucleic acid refers to the order of nucleotides in the polynucleotide, including either or both strands of a double-stranded DNA molecule, e.g., the sequence of both the coding strand and its complement, or of a single-stranded nucleic acid molecule. For example, in alternative embodiments, the promoter of the invention comprises untranscribed, untranslated, and intronic TERT sequences, e.g., as set forth in the exemplary SEQ ID NO:05 and SEQ ID NO:06.

As used herein, the term “transcribable sequence” refers to any sequence which, when operably linked to a cis-acting transcriptional control element, e.g., a promoter, such as the TERT promoters of the invention, and when placed in the appropriate conditions, is capable of being transcribed to generate RNA, e.g., messenger RNA (mRNA).

The term “assessing” includes any form of measurement, and includes determining if an element is present or not. The terms “determining”, “measuring”, “evaluating”, “assessing” and “assaying” are used interchangeably and include quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

The terms “identical” or percent “identity,” in the context of two or more nucleic acids or polypeptide sequences, refer to two or more sequences or subsequences that are the same or have a specified percentage of nucleotides (or amino acid residues) that are the same, when compared and aligned for maximum correspondence over a comparison window, as measured using one of the following sequence comparison algorithms or by manual alignment and visual inspection. This definition also refers to the complement of a sequence. For example, in alternative embodiments, nucleic acids within the scope of the invention include those with a nucleotide sequence identity that is at least about 60%, at least about 75-80%, about 90%, and about 95% of the exemplary TERT promoter sequence set forth in SEQ ID NO:05 (including residues 44 to 13544 of SEQ ID NO:05) or SEQ ID NO:06. Two sequences with these levels of identity are “substantially identical.” Thus, if a sequence has the requisite sequence identity to a TERT promoter sequence or subsequence of the invention, it also is a TERT promoter sequence within the scope of the invention. Preferably, the percent identity exists over a region of the sequence that is at least about 25 nucleotides in length, more preferably over a region that is at least about 50-100 nucleotides in length.

For sequence comparison, typically one sequence acts as a reference sequence, to which test sequences are compared. When using a sequence comparison algorithms test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identity for the test sequence(s) relative to the reference sequence, based on the designated or default program parameters. A “comparison window”, as used herein, includes reference to a segment of any one of the number of contiguous positions selected from the group consisting of from 25 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendrogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence (e.g., a TERT promoter sequence of the invention as set forth by. e.g., SEQ ID NO:05 or SEQ ID NO:06) is compared to another sequence to determine the percent sequence identity relationship (i.e., that the second sequence is substantially identical and within the scope of the invention) using the following parameters: default gap weight (3.00), default gap length weight (0.10), and weighted end gaps. PILEUP can be obtained from the GCG sequence analysis software package, e.g., version 7.0 (Devereaux (1984) Nuc. Acids Res. 12:387-395).

Another example of algorithm that is suitable for determining percent sequence identity (i.e., substantial similarity or identity) is the BLAST algorithm, which is described in Altschul (1990) J. Mol. Biol. 215:403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score fbr mismatching residues, always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In one embodiment, to determine if a nucleic acid sequence is within the scope of the invention, the BLASTN program (for nucleotide sequences) is used incorporating as defaults a wordlength (W) of 11, an expectation (E) of 10, M=5, N=4, and a comparison of both strands. For amino acid sequences, the BLASTP program uses as default parameters a wordlength (W) of 3, an expectation (E) of 10, and the BLOSUM62 scoring matrix (see, e.g., Henikoff (1989) Proc. Natl. Acad. Sci. USA 89:10915).

The BLAST algorithm also performs a statistical analysis of the similarity between two sequences (see, e.g., Karlin (1993) Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### DESCRIPTION OF THE SPECIFIC EMBODIMENTS

Methods and compositions for assaying an agent for TERT promoter modulatory activity are provided. In the subject methods, an agent is contacted with an expression system that includes a TERT promoter nucleic acid operably linked to a heterologous reporter nucleic acid. A feature of the expression system is that it is integrated into a carrier nucleic acid, e.g., a chromosome, in a manner such that it is known to be inactive under wild-type conditions but not positionally silenced. Also provided are compositions, systems and kits thereof, as well as devices, that find use in practicing the subject methods. The subject invention finds use in assaying agents for TERT promoter modulatory activity, such as in a high throughput format.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Although any methods, devices and materials similar or equivalent to those described herein can be used in the practice or testing of the invention, the preferred methods, devices and materials are now described.

All publications mentioned herein are incorporated herein by reference for the purpose of describing and disclosing the elements that are described in the publications which might be used in connection with the presently described invention.

In further describing the invention, the subject methods are described first in greater detail, followed by a review of representative applications in which the subject methods find use, as well as a discussion of representative systems and kits that find use in practicing the subject methods.

Methods

As summarized above, the subject invention provides methods of determining whether an agent has TERT promoter modulatory activity. An agent is considered to have TERT promoter modulatory activity if its interaction with TERT promoter causes a change in transcription activity, e.g., level (for example, in terms of transcribed copies of a coding sequence for a given period of time) of a nucleic acid sequence (e.g., a transcribable sequence) operably linked to the promoter, e.g., as compared to a control (e.g., the transcription activity of an analogous TERT promoter/reporter nucleic acid construct not contacted with the agent of interest). The change that is observed may be an increase or decrease of transcription of the operably linked nucleic acid. In other words, the agent may enhance or inhibit transcription of the nucleic acid sequence operably linked to the TERT promoter. By enhance is meant that the expression level of the operably linked reporter nucleic acid sequence is increased by at least about 2 fold, usually by at least about 5 fold and sometimes by at least 25, 50, 100 fold and in particular about 300 fold or higher, as compared to a control, i.e., expression from an analogous or identical expression system that is not contacted with the agent in question. Alternatively, in cases where expression of the operably linked nucleic acid is so low that it is undetectable, expression of the operably linked nucleic acid is considered to be enhanced if expression is increased to a level that is easily detectable By inhibit is meant that the expression level of the operably linked nucleic acid sequence is decreased by at least about 2 fold, usually by at least about 5 fold and sometimes by at least 25, 50, 100 fold and in particular about 300 fold or higher, as compared to a control, i.e., expression from an analogous or identical expression system that is not contacted with the agent in question. Alternatively, in cases where expression of the operably linked nucleic acid is detectable, expression of the operably linked nucleic acid is considered to be inhibited if expression is decreased to a level that is not detectable.

In practicing the subject methods, an agent to be tested or assayed for TERT promoter modulatory activity (sometimes referred to herein as a candidate agent) is contacted with an expression system that includes a TERT promoter domain (i.e., sequence or region) operably linked to a reporter nucleic acid. In the expression systems employed in the subject methods, the TERT promoter domain or region may be a sequence as described above in the definitions section.

In certain embodiments, the TERT promoter domain of the employed expression systems does not include the full sequence of a TERT minimal promoter, e.g., the human TERT minimal promoter. The full sequence of the human TERT minimal promoter appears as SEQ ID NO:07. In these embodiments, the subject nucleic acids include no more than about 90 number %, usually no more than about 80 number % and more usually no more than about 75 number %, where in many embodiments the subject nucleic acids include less than about 50 number %, sometimes less than about 40 number % and sometimes less than about 25 number % of the total sequence of the a TERT minimal promoter, e.g., the hTERT minimal promoter. In certain embodiments, the length of the TERT promoter domain ranges from about 5 to about 5000 bases, such as.from about 10 to about 2500 bases and including from about 10 to about 1000 bases, where in certain embodiments the length ranges from about 10 to about 500 bases, such as from about 10 to about 250 bases and including from about 10 to about 100 bases, e.g., from about 10 to about 50 bases.

The TERT promoter domain, region or sequence, may include a nucleic acid sequence found in any TERT promoter of interest. In many embodiments, the TERT promoter of interest will be mammalian, where representative specific TERT promoters of interest include the human and mouse TERT promoters.

In representative embodiments of interest, the TERT promoter domain is a human TERT promoter that includes a Site C repressor binding site. In these embodiments, the Site C repressor site does not exceed 20 bases in length, and typically ranges in length from about 7 bases to about 20 bases, and is typically from about 8 bases to about 17 bases long. In certain embodiments, the length of the Site C repressor site/domain ranges from about 14 to about 16 bases, and is often 15 bases long. In certain embodiments, the Site C site has a sequence found in a limited region of the human TERT minimal promoter, where this limited region typically ranges from about −40 to about −90, usually from about −45 to about −85 and more usually from about −40 to about −70 relative to the “A” base or residue of the telomerase ATG codon. In certain embodiments, the Site C repressor site found in the TERT promoter domain of the subject expression systems employed in representative embodiments of the subject methods comprises a sequence of nucleotide residues that is bound by an E2F protein, or at least an E2F DNA binding domain of an E2F protein or other protein that includes such a binding domain. E2F proteins to which the subject Site C repressor site binds include, but are not limited to: E2F-1, E2F-2, E2F-3, E2F-4, E2F-5 and E2F-6. In certain embodiments, the E2F binding domain of the subject repressor sites is the binding. domain as described in Nevins, Science 1992 Oct 16; 258(5081): 424-9. In certain of these embodiments, the Site C site has a sequence that matches the consensus sequence TTTSGCGC, where S is G or C. A sequence is considered to match this consensus sequence if it shares sequence identity with at least 5 of the residues, typically with at least 6 of the residues. In certain of these embodiments, the E2F binding domain found in the Site C sequence is: TTTCAGGC.

Of particular interest in certain embodiments is a nucleic acid having the following sequence:

| | |
|---|---|
| XCGAGTTTCAGGCAG; | (SEQ ID NO:01) |

wherein:
   X is G, A or T, but not C.

As such, specific sequences of interest are:

| | |
|---|---|
| GCGAGTTTCAGGCAG; | (SEQ ID NO:02) |
| ACGAGTTTCAGGCAG; | (SEQ ID NO:03) |
| and | |
| TCGAGTTTCAGGCAG. | (SEQ ID NO:04) |

In certain embodiments, the target Site C site includes the sequence of −66 to −52 of the human TERT minimal promoter. In other words, the sequence of the Site C site is:

| | |
|---|---|
| GCGAGTTTCAGGCAG. | (SEQ ID NO:02) |

In other embodiments, the subject nucleic acids have a sequence that is substantially the same as, or identical to, the Site C repressor binding site sequences as described above, e.g., SEQ ID NOs: 01 to 04. A given sequence is considered to be substantially similar to this particular sequence if it shares high sequence similarity with the above-described specific sequences, e.g., at least 75% sequence identity, usually at least 90%, more usually at least 95% sequence identity with the above specific sequences. Sequence similarity is calculated based on a reference sequence, which may be a subset of a larger sequence. A reference sequence will usually be at least about 18 nucleotides long, more usually at least about 30 nucleotides long, and may extend to the complete sequence that is being compared. Algorithms for sequence analysis are known in the art, such as BLAST, described in Altschul et al. (1990), J. Mol. Biol. 215:403-10 (using default settings, i.e. parameters w=4 and T=17). Of particular interest in certain embodiments are nucleic acids of substantially the same length as the specific nucleic acid identified above, where by substantially the same length is meant that any difference in length does not exceed about 20 number %, usually does not exceed about 10 number % and more usually does not exceed about 5 number %; and have sequence identity to this sequence of at least about 90%, usually at least about 95% and more usually at least about 99% over the entire length of the nucleic acid.

In certain embodiments, the TERT promoter domain is a minimized minimal promoter domain. A mimimized minimal promoter domain of the subject invention is a domain that includes the above described Site C domain, and includes no more than about 80 consecutive bases of the TERT minimal promoter, e.g., no more than about 75 consecutive bases of the TERT minimal promoter; where the Site C domain is, in representative embodiments, flanked by domains found in the corresponding minimal promoter that range in length from about 5 to about 70 nucleotides, e.g., from about 5 to about 50 nucleotides. Specific representative minimized minimal promoters of interest include, but are not limited to:

(SEQ ID NO:08)

−107 to −37
CCCTCCCCTTGCTTTCCGCGGCCCCGCCCTCTCCTCGCGGCGCGAGTTTC
AGGCAGCGCTGCGTCCTG

(SEQ ID NO:09)

−107 to −49
CCCTCCCCTTCCTTTCCGCGGCCCCGCCCTCTCCTCGCGGCGCGAGTTTC
AGGCAGCGC

(SEQ ID NO:10)

−107 to −50
CCCTCCCCTTCCTTTCCGCGGCCCCGCCCTCTCCTCGCGGCGCGAGTTTC
AGGCAGCG

(SEQ ID NO:11)

−107 to −51
CCCTCCCCTTCCTTTCCGCGGCCCCGCCCTCTCCTCGCGGCGCGAGTTTC
AGGCAGC

(SEQ ID NO:12)

−106 to −37
CCTCCCCTTCCTTTCCGCGGCCCCGCCCTCTCCTCGCGGCGCGAGTTTCA
GGCAGCGCTGCGTCCTG

(SEQ ID NO:13)

−106 to −49
CCTCCCCTTCCTTTCCGCGGCCCCGCCCTCTCCTCGCGGCGCGAGTTTCA
GGCAGCGC

(SEQ ID NO:14)

−106 to −50
CCTCCCCTTCCTTTCCGCGGCCCCGCCCTCTCCTCGCGGCGCGAGTTTCA
GGCAGCG

(SEQ ID NO:15)

−106 to −51
CCCTCCCCTTCCTTTCCGCGGCCCCGCCCTCTCCTCGCGGCGCGAGTTTC
AGGCAGC

The numbering of the above sequences corresponds to their position in the wild type human minimal promoter relative to the “A” base or residue of the telomerase ATG codon.

As indicated above, the TERT promoter domain of the employed expression systems is operably linked to a reporter nucleic acid, also referred to herein as a transcribable sequence. A reporter nucleic acid may be any nucleic acid whose transcription can be detected and employed to determine activity of the promoter to which it is operably linked. In representative embodiments, the reporter nucleic acid encodes a detectable product, where the product may be directly or indirectly detectable. By “encodes a detectable product” is meant that the reporter nucleic acid is transcribed into an mRNA that may be detected, e.g., by using any convenient mRNA specific probe, where the mRNA may be further translated into a detectable polypeptide, e.g., protein, that may be directly or indirectly detectable. By directly detectable is meant that the product is detectable without interaction with one or more additional members of a signal producing system. By indirectly detectable is meant that the product interacts with one or more additional members of a signal producing system in order to produce a detectable signal. As such, in representative embodiments, the reporter nucleic acid (i.e., the transcribable sequence) can encode a protein that is detectable by fluorescence, phosphorescence, or by virtue of its possessing an enzymatic activity. Representative detectable proteins of interest include directly detectable proteins, e.g., fluorescent proteins, including but not limited to: green fluorescent protein, enhanced green fluorescent protein, reef coral fluorescent proteins, etc.; and indirectly detectable proteins, such as proteins having an enzymatic activity that, in concert with an additional member(s) of a signal producing system, e.g., a chromogenic substrate, produce a detectable signal, where representative examples include, but are not limited to: firefly luciferase, α-glucuronidase, β-galactosidase, β-lactamase, chloramphenicol acetyl transferase, and the human secreted alkaline phosphatase.

As mentioned above, in certain representative embodiments, the transcript of the reporter nucleic acid operably linked to the promoter is the product that is detected, e.g., using any convenient transcript detection protocol. In other words, the reporter nucleic acid is transcribed into a target transcript, and the presence of the target transcript is detected to determine whether or not expression of the reporter nucleic acid of the expression cassette is being repressed.

In certain of these embodiments, the transcript detection protocol that is employed is an amplification protocol. The term “amplification” as used herein incorporates its common usage and refers to the use of any suitable amplification methodology for generating or detecting a recombinant or naturally expressed nucleic acid.

In representative embodiments, the amplification protocol employed to detect the transcript product of the reporter nucleic acid may be an amplification based quantitation method. In representative quantitation methods of the present invention, the transcript mRNA of the reporter nucleic acid of the expression construct, hereinafter referred to as the target mRNA, is amplified using primers specific for the transcript and the rate or amount of product generated is measured in a manner which allows calculation of the initial target copy number. In representative embodiments, the amplification is carried out using a polymerase chain reaction (PCR). Amplification by the polymerase chain reaction (PCR), is described in U.S. Pat. Nos. 4,683,195; 4,683,202; and 4,965,188; each incorporated herein by reference. Examples of the numerous articles published describing methods and applications of PCR and, in particular, quantitative PCR, are found in PCR Applications, 1999, (Innis et al., eds., Academic Press, San Diego), PCR Strategies, 1995, (Innis et al., eds., Academic Press, San Diego); and PCR Protocols, 1990, (Innis et al., eds., Academic Press, San Diego), each incorporated herein by reference. Commercial vendors, such as Perkin Elmer (Norwalk, Conn.) market PCR reagents and publish PCR protocols.

Amplification of RNA can be carried out by first reverse-transcribing the target RNA using, for example, a viral reverse transcriptase, and then amplifying the resulting cDNA. In representative embodiments, amplification of hTERT mRNA is carried out using a combined high-temperature reverse-transcription-polymerase chain reaction (RT-PCR), as described in U.S. Pat. Nos. 5,310,652; 5,322,770; 5,561,058; 5,641,864; and 5,693,517; each incorporated herein by reference (see also Myers and Sigua, 1995, in PCR Strategies, supra, chapter 5).

As the polymerase chain reaction is one represenative amplification method, amplification of target sequences in a sample may be accomplished by any known method suitable for amplifying the target sequence described above. Suitable amplification methods include the strand displacement assay (Walker et al., 1992, Proc. Natl. Acad. Sci. USA 89:392-396, Walker et al. 1992, Nucleic Acids Res. 20:1691-1696, and U.S. Pat. No. 5,455,166) and the transcription-based amplification systems, including the methods described in U.S. Pat. Nos. 5,437,990; 5,409,818; and 5,399,491; the transcription amplification system (TAS) (Kwoh et al., 1989, Proc. Natl. Acad. Sci. USA 86:1173-1177); and self-sustained sequence replication (3SR) (Guatelli et al., 1990, Proc. Natl. Acad. Sci. USA 87:1874-1878 and WO 92/08800); each of which provides sufficient amplification so that the target sequence can be detected. A review of amplification methods is provided in Abramson and Myers, 1993, Current Opinion in Biotechnology 4:4147, incorporated herein by reference.

Any method for quantitatively detecting the amplified product can be used, including, for example, using fluorescent dyes or labeled probes. Suitable assay formats for detecting hybrids formed between probes and target nucleic acid sequences in a sample are known in the art and include the immobilized target assay formats, such as the dot-blot format, and immobilized probe assay formats, such as the reverse dot-blot assay. Dot blot and reverse dot blot assay formats are described in U.S. Pat. Nos. 5,310,893; 5,451,512; and 5,468,613, each incorporated herein by reference.

In a representative probe-based method, quantitation is carried out using a “TaqMan” or “5′-nuclease assay”, as described in U.S. Pat. Nos. 5,210,015; 5,487,972; and 5,804,375; and Holland et al., 1988, Proc. Natl. Acad. Sci. USA 88:7276-7280, each incorporated herein by reference. In the TaqMan assay, labeled detection probes that hybridize within the amplified region are added during the amplification reaction mixture. The probes are modified so as to prevent the probes from acting as primers for DNA synthesis. The amplification is carried out using a DNA polymerase that possesses 5′ to 3′ exonuclease activity, e.g., Tth DNA polymerase. During each synthesis step of the amplification, any probe which hybridizes to the target nucleic acid downstream from the primer being extended is degraded by the 5′ to 3′ exonuclease activity of the DNA polymerase. Thus, the synthesis of a new target strand also results in the degradation of a probe, and the accumulation of degradation product-provides a measure of the synthesis of target sequences.

Any method suitable for quantitatively detecting degradation product can be used in the TaqMan assay. In a preferred method, the detection probes are labeled with two fluorescent dyes, one of which is capable of quenching the fluorescence of the other dye. The dyes are attached to the probe, preferably one attached to the 5′ terminus and the other is attached to an internal site, such that quenching occurs when the probe is in an unhybridized state and such that cleavage of the probe by the 5′ to 3′ exonuclease activity of the DNA polymerase occurs in between the two dyes. Amplification results in cleavage of the probe between the dyes with a concomitant elimination of quenching and an increase in the fluorescence observable from the initially quenched dye. The accumulation of degradation product is monitored by measuring. the increase in reaction fluorescence. U.S. Pat. Nos. 5,491,063 and 5,571,673, both incorporated herein by reference, describe alternative methods for detecting the degradation of probe which occurs concomitant with amplification.

An alternative, probe-less method, referred to herein as a kinetic-PCR method, for measuring the increase in amplified nucleic acid by monitoring the increase in the total amount of double-stranded DNA in the reaction mixture is described in Higuchi et al., 1992, Bio/Technology 10:413-417; Higuchi el al., 1993, Bio/Technology 11:1026-1030; Higuchi and Watson, in PCR Applications, supra, Chapter 16; U.S. Pat. No. 5,994,056; and European Patent Publication Nos. 487,218 and 512,334, each incorporated herein by reference. The detection of double-stranded target DNA relies on the increased fluorescence that ethidium bromide (EtBr) and other DNA-binding dyes exhibit when bound to double-stranded DNA. The increase of double-stranded DNA resulting from the synthesis of target sequences results in an increase in the amount of dye bound to double-stranded DNA and a concomitant detectable increase in fluorescence.

Quantitation of a sample containing an unknown number of target sequences typically is carried out with reference to a “standard curve” generated from a series of amplifications of samples containing the target sequence in a range of known amounts. The standard curve is used to calculate an input copy number from the signal generated during an amplification. Thus, the unknown target sequence copy number in the sample of interest is estimated using the standard curve by calculating the copy number that previously was determined to yield a signal equal to that observed. The concentration of the target sequence in the sample then is calculated from the input copy number and the sample size, which is determined prior to the reaction.

Quantitative estimates can be sensitive to variability in either the input sample size or in the reaction efficiency. The effect of inter-reaction variability of the input sample size on the calculated target transcript concentration can be eliminated by using a control gene. As described in the examples, a control gene is selected which provides an independent measure of the amount of RNA in the sample. The calculated concentration of target transcript is adjusted based on the independent measure of sample size.

Variability in the amplification efficiency between the reactions used to generate the standard curve and the reaction used to assay the sample of interest can affect the applicability of the standard curve. Carrying out the reactions used to generate the standard curve simultaneously with the reaction used to assay the sample of interest, using the same “master mix” of amplification reagents, and, preferably, in adjacent wells in the same thermal cycler, will minimize the inter-reaction variation in efficiency. Alternatively, an internal standard can be used to adjust the results to account for variation in amplification efficiency.

The effect of inter-reaction variability of reaction efficiency between the reactions used to generate the standard curve and the reaction used to assay the sample of interest can be eliminated by using an internal standard. The internal standard is added to reaction in a known copy number and co-amplified along with the target transcript. The signal generated from the known amount of the internal standard provides an indication of the overall reaction efficiency which can be used to adjust the estimated copy number to account for the difference in reaction efficiencies.

Amplification-based quantitation methods using an internal standard are described in U.S. Pat. Nos. 5,219,727 and 5,476,774; incorporated herein by reference. The internal standard is a nucleotide sequence that contains the same primer binding sites present in the target such that it is amplified by the same primer pair, but is distinguishable from the target sequence either by length or by the presence of a unique internal sequence. The internal standard is included in a known copy number amplifications of the sample of interest and is amplified with approximately the same efficiency as the target sequence. Any change in the signal generated by amplification of the internal standard relative to the signal expected from the standard curve reflects a change in the overall reaction efficiency and is used to adjust the estimate of the target sequence copy number correspondingly.

Sample preparation methods suitable for the amplification of RNA are well known in the art and fully described in the literature cited herein. The particular method used is not a critical part of the present invention. One of skill in the art can optimize reaction conditions for use with the known sample preparation methods.

Further representative protocols of interest include those described in U.S. Pat. No. 6,664,046; the disclosure of which is herein incorporated by reference. The overall length of the expression system may vary depending on the particular lengths of the promoter and reporter domains thereof

A feature of the subject invention is that the employed expression system is present in a nucleic acid background in a manner such that it is inactive under wild-type conditions but is not positionally silenced. In other words, the expression system is integrated into a nucleic acid carrier such that its activity is controlled, e.g., repressed or enhanced, by transacting factors, but it is not inoperative because of its integration location into the nucleic acid carrier. While in the broadest sense the nucleic acid carrier may be any convenient nucleic acid, including but not limited to: plasmids, etc., in many embodiments of interest the nucleic acid carrier is a chromosome or portion or region thereof. In these latter embodiments, the expression system may be viewed as being chromosomally integrated in a manner such that it is not positionally silenced.

While not being positionally silenced by its location in the nucleic acid background, e.g., chromosome, the expression cassette is not active under wild type conditions. As such, substantially little, if any, transcription occurs of the transcribable sequence when the expression cassette is present under wild type conditions. By wild type conditions is meant any conditions that are analogous to conditions found in a normal cell of an animal from which the TERT promoter component is obtained or derived, where the TERT promoter component is repressed such that it inhibits transcription of its operably linked transcribable sequence. As such, when the expression system is contacted with the one or more factors present in a wild type cell that represses TERT promoter activity, it is repressed even though it is not positionally silenced. For example, when an expression system of the subject invention comprising a human TERT promoter domain is present in a normal human cell, activity of the expression system is repressed.

A feature of the invention is that the expression system is known to be present in a nucleic acid background such that it is not positionally silenced but is repressed under wild type conditions. Accordingly, the expression system is present in a nucleic acid carrier, e.g., integrated into a chromosome, in a location that has been predetermined to provide for the above described characteristics. Accordingly, the expression system employed in the subject assays is one that has been validated to be repressed under wild type conditions but not positionally silenced, such that when the correct trans acting factor or factors are present, the expression system is active.

An expression system present on a carrier nucleic acid according to the subject invention can be produced using any convenient methodology. A representative methodology is the use of a system that provides for site-specific chromosomal integration, such as the system described in the experimental section below. By using such a system, one can first integrate a constitutively active expression cassette in a chromosome to confirm that the integration site does not positionally silence the expression system, and then integrate the expression system of the present invention into the resultant validated site. For example, one can integrate an expression cassette that has a point mutation in the Site C region that causes the expression system to be active, even under wild type conditions. Of interest are expression cassettes that include a mutation at a region from positions −64 to −66 of the promoter, where in representative embodiments, the mutation is a mutation at position −65, e.g., where the C residue in the wild type sequence is substituted with a different base, e.g., a C→A mutation. See e.g., the specific representative Site C point mutations reported in the Experimental Section of U.S. Pat. No. 6,686,159, the disclosure of which is herein incorporated by reference. For example, SEQ ID NOS: 05 to 12, provided above, can be modified to include a C→A mutation at position −65.

In practicing the subject methods, the expression system is contacted with the candidate agent whose activity is to be tested and transcription of the transcribable sequence is evaluated to determine the TERT promoter modulatory activity of the candidate agent. Contact of the candidate agent and expression system may occur in an in vitro or in vivo format, where both formats are readily developed by those of skill in the art. In vitro formats of interest include both cell free and cell based formats. Cell free formats are formats in which the expression system and the candidate agent are contacted in the absence of the cell, where contact will typically occur in an aqueous medium that at least includes the one or more factors which impart to the media the desired conditions, e.g., wild type conditions where activity of the expression system is repressed or mutant conditions where the expression system is active. Another representative in vitro format is a cell based format, in which contact occurs inside of a cell, where the expression system may be integrated into a chromosome thereof. Depending on the particular assay, the cell may be a normal cell that provides wild type conditions, e.g., a cell that normally lacks telomerase activity, e.g., an MRC5 cell, etc.; or the cell may be mutant cell in which telomerase activity is present. In yet other embodiments, the assay may be in vivo, in which an animal transgenic for the expression system is employed, and a candidate agent is contacted with the animal.

The conditions under which the expression system and the candidate agent are contacted may vary depending on the nature of the assay and the nature of the candidate agent modulatory activity to be determined. For example, where the assay is employed to screen candidate agents for TERT promoter derepression activity, i.e., activation activity, the conditions under which the expression system and the agent are contacted are generally wild type conditions, where the conditions may be described as an environment in which, in the absence of the candidate agent, expression of the operably linked transcribable sequence is repressed. Alternatively, where the assay is employed to screen candidate agents for TERT promoter repression activity, i.e., inhibition activity, the conditions under which the expression system and the agent are contacted are generally mutant conditions, where the conditions may be described as an environment in which, in the absence of the candidate agent, expression of the.operably linked transcribable sequence occurs. As indicated above, the desired conditions may be set up in vitro by combining the various required components in an aqueous medium, or the assay may be carried out in vivo.

Following contact of the candidate agent and the expression system, the activity of the expression system is evaluated or assessed to determine the promoter modulatory activity of the candidate agent. This step of assessing or evaluating activity of the expression system will necessarily vary depending on the nature the transcribable reporter sequence, e.g., whether one detects the presence of the transcribed nucleic acid or the presence and/or activity of the translated product thereof, e.g., as is done where the transcribable sequence encodes an enzymatic product, e.g. luciferase or SEAP. In certain embodiments of interest, activity of the expression system is evaluated by determining activity of the product encoded by the transcribable reporter sequence of the expression system. This step of the subject methods may include either a qualitative or quantitative evaluation of the expression system activity, and may or may not include use of one or more reference or controls, as may be desired.

In certain embodiments, the subject methods are performed in a high throughput (HT) format. In the subject HT embodiments of the subject invention, a plurality of different compounds are simultaneously tested. By simultaneously tested is meant that each of the compounds in the plurality are tested at substantially the same time. Thus, at least some, if not all, of the compounds in the plurality are assayed for their effects in parallel. The number of compounds in the plurality of that are simultaneously tested is typically at least about 10, where in certain embodiments the number may be at least about 100 or at least about 1000, where the number of compounds tested may be higher. In general, the number of compounds that are tested simultaneously in the subject HT methods ranges from about 10 to 10,000, usually from about 100 to 10,000 and in certain embodiments from about 1000 to 5000. A variety of high throughput screening assays for determining the activity of candidate agent are known in the art and are readily adapted to the present invention, including those described in e.g., Schultz (1998) Bioorg Med Chem Lett 8:2409-2414; Weller (1997) Mol Divers. 3:61-70; Fernandes (1998) Curr Opin Chem Biol 2:597-603; Sittampalam (1997) Curr Opin Chem Biol 1:384-91; as well as those described in published United States application 20040072787 and issued U.S. Pat. No. 6,127,133; the disclosures of which are herein incorporated by reference.

Testing of a candidate agent according to the invention as described above readily determines whether or not an agent has TERT promoter modulatory activity. As mentioned above, an agent is considered to have TERT promoter modulatory activity if its interaction with TERT promoter causes a change in transcription activity, e.g., level (for example, in terms of transcribed copies for a given period of time), of a nucleic acid sequence (i.e., transcribable sequence) operably linked to the promoter, e.g., as compared to a control (e.g., the transcription activity of an analogous TERT promoter/reporter nucleic acid construct not contacted with the agent of interest). The change that is observed may be an increase or decrease of transcription of the operably linked nucleic acid. In other words, the agent may enhance or inhibit transcription of the nucleic acid sequence operably linked to the TERT promoter. By enhance is meant that the expression level of the operably linked reporter nucleic acid sequence is increased by at least about 2 fold, usually by at least about 5 fold and sometimes by at least 25, 50, 100 fold and in particular about 300 fold or higher, as compared to a control, i.e., expression from an analogous or identical expression system that is not contacted with the agent in question. Alternatively, in cases where expression of the operably linked nucleic acid is so low that it is undetectable, expression of the operably linked nucleic acid is considered to be enhanced if expression is increased to a level that is easily detectable. By inhibit is meant that the expression level of the operably linked nucleic acid sequence is decreased by at least about 2 fold, usually by at least about 5 fold and sometimes by at least 25, 50, 100 fold and in particular about 300 fold or higher, as compared to a control, i.e., expression from an analogous or identical expression system that is not contacted with the agent in question.

Utility

A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The subject assays find use in any application where it is desired to determine whether a candidate agent has TERT promoter modulatory activity. Specifically, the subject assays find use in applications where one wishes to determine whether an agent has TERT promoter repressor activity and in applications where one wishes to determine whether an agent has TERT promoter activator or enhancer activity.

Agents identified in the above screening assays that inhibit repression of TERT transcription find use in the methods of enhancement of TERT expression, e.g., in the treatment of disease conditions, in research applications, etc., where representative specific applications include those described in United States Published Applications: 20030211965; 20030171326; 20030104420; 20030050264; and 20020193289; the disclosures of which are herein incorporated by reference. Alternatively, agents identified in the above screening assays that enhance repression find use in applications where inhibition of TERT expression is desired, e.g., in the treatment of disease conditions characterized by the presence of unwanted TERT expression, such as cancer and other diseases characterized by the presence of unwanted cellular proliferation, where such methods are described in, for example, U.S. Pat. Nos. 5,645,986; 5,656,638; 5,703,116; 5,760,062; 5,767,278; 5,770,613; and 5,863,936; the disclosures of which are herein incorporated by reference.

Kits

Also provided are kits that find use in practicing the subject methods, as described above. For example, in some embodiments, kits for practicing the subject methods include at least an expression system as described above, where the expression system may be present on a variety of different nucleic acid backgrounds or carriers, including integrated into a chromosome of a cell. Furthermore, additional reagents that are required or desired in the protocol to be practiced with the kit components may be present, which additional reagents include, but are not limited to: aqueous mediums, culture mediums, enzyme substrates or other members of a signal producing system, and the like. The kits may also include reference or control elements, e.g., that provide calibration signals or values for use in assessing the observed signal generated by an assay performed with the kit components. The kit components may be present in separate containers, or one or more of the components may be present in the same container, where the containers may be storage containers and/or containers that are employed during the assay for which the kit is designed.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Systems

Also provided are systems that find use in practicing the subject methods, as described above. For example, in some embodiments, systems for practicing the subject methods include at least an expression system as described above, where the expression system may be present on a variety of different nucleic acid backgrounds or carriers, including integrated into a chromosome of a cell. Furthermore, additional reagents that are required or desired in the protocol to be practiced with the system components may be present, which additional reagents include, but are not limited to: aqueous mediums, culture mediums, enzyme substrates or other members of a signal producing system, and the like. The systems may also include reference or control elements, e.g., that provide calibration signals or values for use in assessing the observed signal generated by an assay performed with the system components. Devices

Also provided are high throughput (HT) devices that find use in practicing the subject methods, particularly HT embodiments thereof. The high throughput devices may have any convenient configuration, and generally include a plurality of two or more fluid containment elements in which assays can take place, agent administration elements and signal detection elements. For example, representative HT devices of the subject invention include a plate or substrate having a plurality of fluid-containing wells, reagent-adding equipment responsive to a computer for adding reagent, e.g., candidate agent, to the wells, measurement equipment for measuring at least one attribute, e.g., fluorescence, of the solution contained by the wells and moving equipment which is responsive to the computer for aligning one of the wells first with the reagent-adding component, then with the measurement device, as further described in U.S. Pat. No. 6,127,133, the disclosure of which is herein incorporated by reference. Also of interest are the devices described in U.S. Pat. No. 6,468,736 and 5,989,835; the disclosures of which are herein incorporated by reference. A feature of the HT devices of the present invention is that they include in at least one fluid containment element an expression system as described above.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES

I. High Throughput Screening System

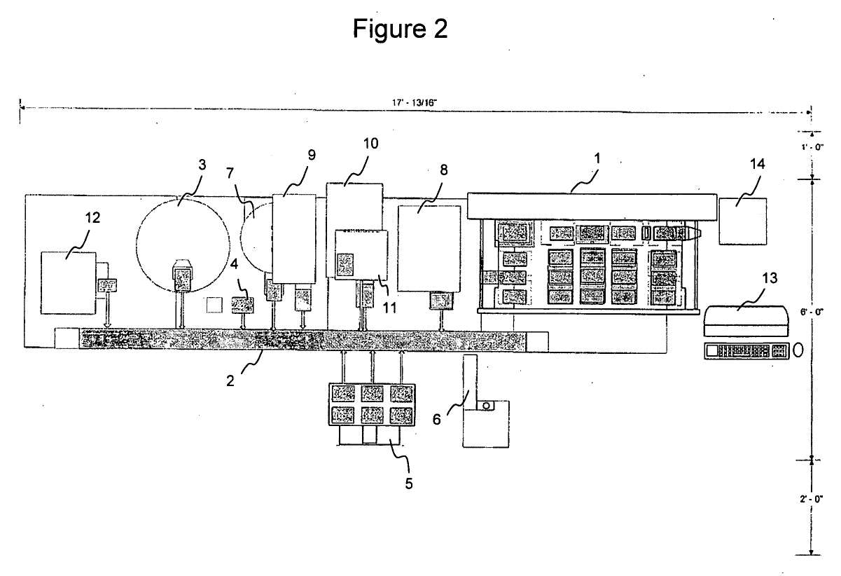

An exemplary high throughput screening system of the invention is configured according to FIG. 2. The elements of FIG. 2 are as follows:
(1) Beckman Coulter Biomek F/X liquid handling station; (2) Beckman Coulter ORCA robotic arm on 3 meter rail; (3) Beckman Coulter labware storage carousel; (4) Beckman Coulter re-grip station with barcode reader; (5) Beckman Coulter tip-lift; (6) Beckman Coulter lid disposal station; (7) Beckman Coulter microtiter plate de-lidding station; (8) Perkin Elmer Envision multimode plate reader; (9) ABgene ALPS-300 plate sealer; (10) ABgene ASP-50 plate piercer; (11) Thermo Electron Multidrop liquid dispenser; (12) Kendro Cytomat 2 incubator with internal robotics; (13) Automation Controller with monitor; (14) Utilities (electric, air, vacuum). The sealer (9) is stacked over the de-lidding station (7) and the plate piercer (10) is stacked over the multidrop (11). Recommended parameters for the Utilities (element 14) include:
   Air: 45 to 60 psi. minimum
   Vacuum: 15-25 inHg, free air capacity of 75 l/min.
   Electrical: four 15 Amp circuits or three 20 Amp circuits

The companies that manufacture the components depoecited in FIG. 2 are Beckman Coulter, Fullerton, Calif.; Perkin Elmer, Wellesley, Mass.; ABgene, Surrey, United Kingdom, Thermo Electron Corporation, Waltham, Mass.; and Kendro Laboratory Products, Lane Cove, Australia.

II. Minimized Minimal Promoters

In order to better understand the repression mechanisms of the hTERT promoter, the minimal promoter (MP; SEQ ID NO. 7) was reduced in size to identify the minimum unit of DNA that can still function in transcription initiation and still be repressible at Site C. The results of these experiments are shown in Table 1, below.

TABLE 1

| | | | Promoter | | Fold | |
|---|---|---|---|---|---|---|
| | | Point | Strength | | Repression | |
| pSSI # | Region(s) | Mutation | MRC5 | HELA | MRC5 | HELA |
| 509 | −258 to −1 | None | | 100.0% | | |
| 673 | −258 to −1 | −65 C−>A | 100.0% | 100.0% | 13.408 | 1.155 |
| 1331 | −107 to −37 | None | | 45.2% | | |
| 1487 | −107 to −37 | −65 C−>A | 31.8% | 55.2% | 8.803 | 0.975 |
| 1635 | −105 to −37 | None | | 5.8% | | |
| 1631 | −105 to −37 | −65 C−>A | 0.4% | 1.8% | | |
| 1520 | −107 to −49 | None | | 18.5% | | |
| 1523 | −107 to −49 | −65 C−>A | 10.2% | 16.9% | 4.275 | 1.067 |

The PSSI# is the name of the promoter/reporter plasmid containing each MMP. The next column shows the region, in base numbers relative to the initiating ATG codon, of the telomerase promoter containing each MMP (see SEQ ID NO. 7). The next column indicates the presence or absence of a site-specific mutation in the MMP that prevents repression (i.e., changing nucleotide −65 from C to A). The next two columns show the promoter strength of each MMP relative to the MP. A score of 100% in this column means that the expression level of the reporter gene, secreted alkaline phosphatase (SEAP), that was detected in a transient transfection assay of the MMP was as high as the level of SEAP detected from the MP. In all cases the promoter strength is relative to the MP containing the same mutations as he MMP. That is, if the MMP contains the −65 C to A mutation, its promoter strength was measured relative to the MP containing the −65 C to A mutation. Promoter strengths are shown for each MMP in two different cell lines, MRC5 and HELA. MRC5 is a normal mortal cell line that represses the telomerase promoter. HELA is an immortal cell line that does not repress the telomerase promoter. In the cases of plasmids that do not contain the −65 C to A mutation promoter strengths in MRC5 are not shown. This is because the level of SEAP detected when using the MP and the MMP was very low, and as such, the promoter strengths were essentially zero divided by zero. The next two columns show the “Fold Repression” of each plasmid. That is, the level of SEAP for each mutated (i.e., −65 C to A) plasmid divided by the SEAP level for the comparable unmutated plasmid. In cases where the promoter strength was very low, the “Fold Repression” is not shown because the score would essentially be zero divided by zero.

The above results show that when the MP (bases −258 to −1) is reduced to 71 bases (−107 to −37) the promoter still retains 31.8% of its promoter strength in MRC5 and at least 45.2% in HELA. Furthermore, the promoter can still be repressed 8.8 fold in MRC5. When two additional bases are removed from the upstream end (−105 to −37) the promoter strength drops to 0.4% in MRC5 and 1.8% to 5.8% in HELA. This shows that the upstream end of the hTERT promoter that can still function in both activity and repression is either −107 or −106. Regarding the downstream end of the hTERT promoter, when the promoter was shortened to 59 bases (−107 to −49) the promoter still retained 10.2% of its strength in MRC5 and 16.9% to 18.5% of its strength in HELA as well as retaining 4.275 fold repression ability in MRC5. This shows that the downstream end of the telomerase promoter that can still function in transcription initiation and still be repressible at Site C is between −49 and −51 (the downstream end of Site C).

Ill. Generation of Reporter Cell Lines for Use in High-Throughput Assay

Plasmids

pcDNA5/FRT, pFRT/LacZeo and pOG44 were obtained from Invitrogen. A detailed description of these vectors and their use in creating stable cell lines can be found on the Invitrogen website (Invitrogen.com). The use of these plasmids in practicing an exemplary embodiment of the present invention is described below and shown in FIG. 4.

pSS13644 contains the SEAP reporter gene under transcriptional control of the hTERT minimal promoter and includes the FRT/Hygromycin cassette from pcDNA5/FRT. This plasmid also contains an Ampicillin resistance gene and the pUC origin of replication for bacterial amplification (see FIG. 4).

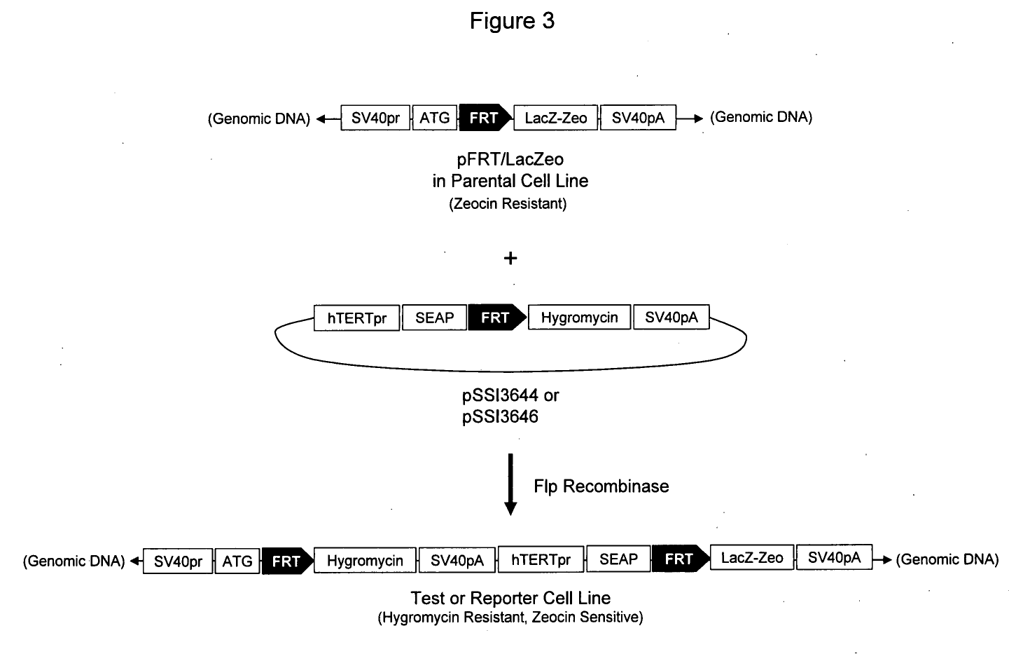

pSS13646 is identical to pSS13644 except that the hTERT minimal promoter has been mutagenized in Site C such that repression of transcription is prevented (i.e., −65 C→A; see FIG. 3). As discussed, this mutation allows transcription from the hTERT promoter in cells in which the wild type hTERT promoter is not transcribed (e.g., most normal cells, including the MRC5 cells shown in FIG. 3).

Generation of Parental Cell Line Containing FRT Recombination Site

CCD18Lu cells (having 45-50 remaining population doublings; available from ATCC) were stably transfected with plasmid pFRT/LacZeo (Invitrogen). The FRT recombination site of pFRT/LacZeo is preceded by an initiator ATG and followed by an ATG-less LacZ-Zeocin™ fusion gene that is in-frame with the initiator ATG (see FIG. 4). The FRT/LacZeo gene is under the control of the constitutive SV40 transcriptional promoter and is followed by a SV40 polyadenylation signal. Zeocin™-resistant clones were isolated and expanded by standard methods (i.e., limiting dilution culture) and screened to identify those clones containing single integrations of the pFRT/LacZeo plasmid (as determined by Southern Blotting and semi-quantitative PCR).

Screening of Parental Cell Lines

Independent clonal pFRT/LacZeo parental cell lines generated in the previous section were screened by transfection with pSS13646 and a plasmid encoding the Flp recombinase (pOG44). As mentioned above, pSSI3646 contains the FRT/Hygromycin cassette from pcDNA5/FRT and the SEAP gene under the control of a mutant hTERT promoter that is active in normal cells. The FRT recombinase site is immediately upstream of a Hygromycin gene that lacks an initiatior ATG (see FIG. 4). When productive recombination occurs between the integrated FRT site in the parental cell line and pSS13646 (in the presence of Flp recombinase expressed from pOG44), the Hygromycin gene is placed downstream of the SV40 promoter and initiator ATG from the FRT/LacZeo cassette and is therefore transcribed and translated (see FIG. 4). The cell line thus acquires Hygromycin resistance. In addition, the hTERT promoter/SEAP cassette is integrated into the parental cell line genome (see FIG. 4).

Hygromycin resistant clones were isolated from each of the transfected parental cell lines and screened for proper integration of pSS13646 into the FRT site using Southern Blotting and PCR analyses. Several positive clonal lines (i.e., test cell lines) from each of the transfected parental cell lines were identified. These test cell lines were then screened for the expression of SEAP. The parental cell lines which gave rise to test cell lines that expressed significant (i.e., detectible) levels of SEAP were designated as having suitable integration sites. Specifically, the integration site of the pFRT/LacZeo in each of these parental cell lines is in a location that is not positionally silenced. As such, reporter cell lines for use in the high throughput assays of the invention can be generated from these parental cell lines.

Generation of Reporter Cell Lines

The clonal parental cell lines designated as suitable in the previous section were transfected with pSS13644 and pOG44. Hygromycin resistant clones were isolated from each of the transfected parental cell lines and screened for proper integration of the pSS13644 plasmid into the FRT site using Southern Blotting and PCR analyses. Several positive clones from each of the parental lines were identified and then screened for the expression or SEAP. In this case, cell lines that expressed significantly reduced levels of SEAP as compared to the same parental cell line clones transfected with pSS13646 (i.e., test cell lines) were chosen as suitable reporter cell lines to use in the high throughput assays of the invention.

As discussed in previous sections, compounds that elicit an increase in the expression of the reporter gene (e.g., SEAP) in a reporter cell line are of particular interest because they have the ability to increase hTERT promoter activity even when the repressor binding site (i.e., Site C) is present.

It is evident from the above results and discussion that the subject invention provides for greatly improved assays for determining the TERT promoter modulatory activity of a candidate agent. Accordingly, the subject invention represents a significant contribution to the art.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of determining whether an agent modulates transcription control activity of a TERT promoter, said method comprising:
(a) contacting said agent with an expression system comprising a TERT promoter nucleic acid operably linked to a heterologous reporter nucleic acid, wherein said expression system is integrated into a nucleic acid in a manner such that it is known to be inactive under wild-type conditions but not positionally silenced; and
(b) evaluating transcription of said heterologous reporter nucleic acid to determine whether said agent modulates transcription control activity of said TERT promoter nucleic acid.

2. The method according to claim 1, wherein said contacting step (a) occurs under wild-type conditions and said method is a method of determining whether said agent enhances expression controlled by a TERT promoter nucleic acid.

3. The method according to claim 1, wherein said contacting step (a) occurs under conditions in which expression of said operably linked heterologous reporter nucleic acid is not repressed and said method is a method for determining whether said agent inhibits expression controlled by a TERT promoter nucleic acid.

4. The method according to claim 1, wherein said TERT promoter nucleic acid is a human TERT promoter nucleic acid.

5. The method according to claim 1, wherein said expression system is present in a cell free environment.

6. The method according to claim 1, wherein said expression system is present inside of a cell.

7. The method according to claim 1, wherein transcription of said reporter nucleic acid produces a directly detectable product.

8. The method according to claim 7, wherein said directly detectable product is fluorescent.

9. The method according to claim 1, wherein transcription of said reporter nucleic acid produces an indirectly detectable product.

10. The method according to claim 9, wherein said indirectly detectable product comprises an enzymatic activity.

11. The method according to claim 1, wherein said evaluating comprises comparing detected transcription of said heterologous reporter nucleic acid to a control.

12. The method according to claim 11, wherein said detected transcription is detected by detecting the presence of a transcript of said reporter nucleic acid.

13. The method according to claim 12, wherein said transcript of said reporter nucleic acid is detected using an amplification protocol.

14. The method accordingto claim 13, wherein said amplification protocol is an RT-PCT protocol.

15. The method according to claim 1, wherein said method comprises determining the modulatory activity of at least two different agents.

16. The method according to claim 15, wherein said method is a high-throughput method.

17. The method according to claim 1, wherein said agent is a small molecule.

18-26. (canceled)

27. An expression construct comprising a TERT promoter nucleic acid operably linked to a heterologous reporter nucleic acid, wherein said expression construct is integrated into a nucleic acid in a manner such that it is known to be inactive under wild-type conditions but not positionally silenced.

28-32. (canceled)

33. A cell comprising an expression construct comprising a TERT promoter nucleic acid operably linked to a heterologous reporter nucleic acid, wherein said expression construct is integrated into a chromosome in a manner such that it is known to be inactive under wild-type conditions but not positionally silenced.

34-42. (canceled)

43. A system for determining whether an agent modulates transcription control activity of a TERT promoter, said system comprising:
(a) an expression system comprising a TERT promoter nucleic acid operably linked to a heterologous reporter nucleic acid, wherein said expression system is integrated into a nucleic acid in a manner such that it is known to be inactive under wild-type conditions but not positionally silenced; and
(b) said agent.

44-46. (canceled)


SEQUENCE LISTING (15 sequences)

SEQ ID NO: 1 - DNA, 15 bp, human
  misc_feature 1: n = A,T,C or G
ncgagtttcaggcag

SEQ ID NO: 2 - DNA, 15 bp, human
gcgagtttcaggcag

SEQ ID NO: 3 - DNA, 15 bp, human
acgagtttcaggcag

SEQ ID NO: 4 - DNA, 15 bp, human
tcgagtttcaggcag

SEQ ID NO: 5 - DNA, 15418 bp, human
gcggccgcgagctctaatacgactcactatagggcgtcgactcgatcaatggaagatgaggcattgccgaagaaaagattaatggatttgaacacacagcaacagaaactacatgaagtgaaacacaggaaaaaaaagataaagaaacgaaaagaaaagggcatcagtgagcttcagcagaagttccatcggccttacatatgtgtaagcagaggccctgtaggagcagaggcagggggaaaatactttaagaaataatgtctaaaagtttttcaaatatgaggaaaaacataaaaccacagatccaagaagctcaacaaaacaaagcacaagaaacaggaagaaattaaaagttatatcacagtcaaattgctgaaaaccagcaacaaagagaatatcttaagagtatcagaggaaaagagattaatgacaggccaagaaacaatgaaaacaatacagatttcttgtaggaaacacaagacaaaagacattttttaaaaccaaaaggaaaaaaaatgctacattaaaatgttttttacccactgaaagtatatttcaaaacatattttaggccaggcttggtggctcacacctgtaatcccagcactttgggaggccaaggtgggtggatcgcttaaggtcaggagttcgagaccagcctggccaatatagcgaaaccccatctgtactaaaaacacaaaaattagctgggtgtggtgacacatgcctgtaatcccaggtactcaggaggctaaggcaggagaattgcttgaactgggaggcagaggtggtgagccaagattgcaccagtgcactccagccttggtgacagagtgaaactccatctcaaaaacaaacaaacaaaatacatatacataaatatatatgcacatatatatacatatataaatatatatacacatatataaatctatatacatatatacatatatacacatatataaatctatatacatatatatacatatataatatatttacatatataaatatatacatatataaatatacatatataaatacatatataaatatacatatataaatatacatatataaatatacatatataaatatatacatatataaatatacatatataaatatatatacatatataaatatataaatatacaagtatatacaaatatatacatatataaatgtatatacgtatatacatatatatataaatatataaaaaaacttttggctgggcacctttccaaatctcatggcacatataagtctcatggtaacctcaaataaaaaaacatataacagatacaccaaaaataaaaaccaataaattaaatcatgccaccagaagaaattaccttcactaaaaggaacacaggaaggaaagaaagaaggaagagaagaccatgaaacaaccagaaaacaaacaacaaaacagcaggagtaattcctgacttatcaataataatgctgggtgtaaatggactaaactctccaatcaaaagacatagagtggctgaatggacgaaaaaaacaagactcaataatctgttgcctacaagaatatacttcacctataaagggacacatagactgaaaataaaaggaaggaaaaatattctatgcaaatggaaaccaaaaaaagaacagaactagctacacttatatcagacaaaatagatttcaagacaaaaagtacaaaaagagacaaagtaattatataataataaagcaaaaagatataacaattgtgaatttatatgcgcccaacactgggacacccagatatatacagcaaatattattagaactaaggagagagagagatccccatacaataatagctggagacttcaccccgcttttagcattggacagatcatccagacagaaaatcaaccaaaaaattggacttaatctataatatagaacaaatgtacctaattgatgtttacaagacatttcatccagtagttgcagaatatgcattttttcctcagcatatggatcattctcaaggatagaccatatattaggccacagaacaagccattaaaaattcaaaaaaattgagccaggcatgatggcttatgcttgtaattacagcactttggggagggtgaggtgggaggatgtcttgagtacaggagtttgagaccagcctgggcaaaatagtgagaccctgtctctacaaacttttttttttaattagccaggcatagtggtgtgtgcctgtagtcccagctacttaggaggctgaagtgggaggatcacttgagcccaagagttcaaggctacggtgagccatgattgcaacaccacacaccagccttggtgacagaatgagaccctgtctcaaaaaaaaaaaaaaaaattgaaataatataaagcatcttctctggccacagtggaacaaaaccagaaatcaacaacaagaggaattttgaaaactatacaaacacatgaaaattaaacaatatacttctgaataaccagtgagtcaatgaagaaattaaaaaggaaattgaaaaatttatttaagcaaatgataacggaaacataacctctcaaaacccacggtatacagcaaaagcagtgctaagaaggaagtttatagctataagcagctacatcaaaaaagtagaaaagccaggcgcagtggctcatgcctgtaatcccagcactttgggaggccaaggcgggcagatcgcctgaggtcaggagttcgagaccagcctgaccaacacagagaaaccttgtcgctactaaaaatacaaaattagctgggcatggtggcacatgcctgtaatcccagctactcgggaggctgaggcaggataaccgcttgaacccaggaggtggaggttgcggtgagccgggattgcgccattggactccagcctgggtaacaagagtgaaaccctgtctcaagaaaaaaaaaaaagtagaaaaacttaaaaatacaacctaatgatgcaccttaaagaactagaaaagcaagagcaaactaaacctaaaattggtaaaagaaaagaaataataaagatcagagcagaaataaatgaaactgaaagataacaatacaaaagatcaacaaaattaaaagttggttttttgaaaagataaacaaaattgacaaacctttgcccagactaagaaaaaaggaaagaagacctaaataaataaagtcagagatgaaaaaagagacattacaactgataccacagaaattcaaaggatcactagaggctactatgagcaactgtacactaataaattgaaaaacctagaaaaaatagataaattcctagatgcatacaacctaccaagattgaaccatgaagaaatccaaagcccaaacagaccaataacaataatgggattaaagccataataaaaagtctcctagcaaagagaagcccaggacccaatggcttccctgctggattttaccaatcatttaaagaagaatgaattccaatcctactcaaactattctgaaaaatagaggaaagaatacttccaaactcattctacatggccagtattaccctgattccaaaaccagacaaaaacacatcaaaaacaaacaaacaaaaaaacagaaagaaagaaaactacaggccaatatccctgatgaatactgatacaaaaatcctcaacaaaacactagcaaaccaaattaaacaacaccttcgaaagatcattcattgtgatcaagtgggatttattccagggatggaaggatggttcaacatatgcaaatcaatcaatgtgatacatcatcccaacaaaatgaagtacaaaaactatatgattatttcactttatgcagaaaaagcatttgataaaattctgcacccttcatgataaaaaccctcaaaaaaccaggtatacaagaaacatacaggccaggcacagtggctcacacctgcgatcccagcactctgggaggccaaggtgggatgattgcttgggcccaggagtttgagactagcctgggcaacaaaatgagacctggtctacaaaaaacttttttaaaaaattagccaggcatgatggcatatgcctgtagtcccagctagtctggaggctgaggtgggagaatcacttaagcctaggaggtcgaggctgcagtgagccatgaacatgtcactgtactccagcctagacaacagaacaagaccccactgaataagaagaaggagaaggagaagggagaaaggagggagaagggaggaggaggagaaggaggaggtggaggagaagtggaaggggaaggggaagggaaagaggaagaagaagaaacatatttcaacataataaaagccctatatgacagaccgaggtagtattatgaggaaaaactgaaagcctttcctctaagatctggaaaatgacaagggcccactttcaccactgtgattcaacatagtactagaagtcctagctagagcaatcagataagagaaagaaataaaaggcatccaaactggaaaggaagaagtcaaattatcctgtttgcagatgatatgatcttatatctggaaaagacttaagacaccactaaaaaactattagagctgaaatttggtacagcaggatacaaaatcaatgtacaaaaatcagtagtatttctatattccaacagcaaacaatctgaaaaagaaaccaaaaaagcagctacaaataaaattaaacagctaggaattaaccaaagaagtgaaagatctctacaatgaaaactataaaatattgataaaagaaattgaagagggcacaaaaaaagaaaagatattccatgttcatagattggaagaataaatactgttaaaatgtccatactacccaaagcaatttacaaattcaatgcaatccctattaaaatactaatgacgttcttcacagaaatagaagaaacaattctaagatttgtacagaaccacaaaagacccagaatagccaaagctatcctgaccaaaaagaacaaaactggaagcatcacattacctgacttcaaattatactacaaagctatagtaacccaaactacatggtactggcataaaaacagatgagacatggaccagaggaacagaatagagaatccagaaacaaatccatgcatctacagtgaactcatttttgacaaaggtgccaagaacatactttggggaaaagataatctcttcaataaatggtgctggaggaactggatatccatatgcaaaataacaatactagaactctgtctctcaccatatacaaaagcaaatcaaaatggatgaaaggcttaaatctaaaacctcaaactttgcaactactaaaagaaaacaccggagaaactctccaggacattggagtgggcaaagacttcttgagtaattccctgcaggcacaggcaaccaaagcaaaaacagacaaatgggatcatatcaagttaaaaagcttctgcccagcaaaggaaacaatcaacaaagagaagagacaacccacagaatgggagaatatatttgcaaactattcatctaacaaggaattaataaccagtatatataaggagctcaaactactctataagaaaaacacctaataagctgattttcaaaaataagcaaaagatctgggtagacatttctcaaaataagtcatacaaatggcaaacaggcatctgaaaatgtgctcaacaccactgatcatcagagaaatgcaaatcaaaactactatgagagatcatctcaccccagttaaaatggcttttattcaaaagacaggcaataacaaatgccagtgaggatgtggataaaaggaaacccttggacactgttggtgggaatggaaattgctaccactatggagaacagtttgaaagttcctcaaaaaactaaaaataaagctaccatacagcaatcccattgctaggtatatactccaaaaaagggaatcagtgtatcaacaagctatctccactcccacatttactgcagcactgttcatagcagccaaggtttggaagcaacctcagtgtccatcaacagacgaatggaaaaagaaaatgtggtgcacatacacaatggagtactacgcagccataaaaaagaatgagatcctgtcagttgcaacagcatggggggcactggtcagtatgttaagtgaaataagccaggcacagaaagacaaacttttcatgttctcccttacttgtgggagcaaaaattaaaacaattgacatagaaatagaggagaatggtggttctagaggggtgggggacagggtgactagagtcaacaataatttattgtatgttttaaaataactaaaagagtataattgggttgtttgtaacacaaagaaaggataaatgcttgaaggtgacagataccccatttaccctgatgtgattattacacattgtatgcctgtatcaaaatatctcatgtatgctatagatataaaccctactatattaaaaattaaaattttaatggccaggcacggtggctcatgtccataatcccagcactttgggaggccgaggcggtggatcacctgaggtcaggagtttgaaaccagtctggccaccatgatgaaaccctgtctctactaaagatacaaaaattagccaggcgtggtggcacatacctgtagtcccaactactcaggaggctgagacaggagaattgcttgaacctgggaggcggaggttgcagtgagccgagatcatgccactgcactgcagcctgggtgacagagcaagactccatctcaaaacaaaaacaaaaaaaagaagattaaaattgtaatttttatgtaccgtataaatatatactctactatattagaagttaaaaattaaaacaattataaaaggtaattaaccacttaatctaaaataagaacaatgtatgtggggtttctagcttctgaagaagtaaaagttatggccacgatggcagaaatgtgaggagggaacagtggaagttactgttgttagacgctcatactctctgtaagtgacttaattttaaccaaagacaggctgggagaagttaaagaggcattctataagccctaaaacaactgctaataatggtgaaaggtaatctctattaattaccaataattacagatatctctaaaatcgagctgcagaattggcacgtctgatcacaccgtcctctcattcacggtgctttttttcttgtgtgcttggagattttcgattgtgtgttcgtgtttggttaaacttaatctgtatgaatcctgaaacgaaaaatggtggtgatttcctccagaagaattagagtacctggcaggaagcaggtggctctgtggacctgagccacttcaatcttcaagggtctctggccaagacccaggtgcaaggcagaggcctgatgacccgaggacaggaaagctcggatgggaaggggcgatgagaagcctgcctcgttggtgagcagcgcatgaagtgcccttatttacgctttgcaaagattgctctggataccatctggaaaaggcggccagcgggaatgcaaggagtcagaagcctcctgctcaaacccaggccagcagctatggcgcccacccgggcgtgtgccagagggagaggagtcaaggcacctcgaagtatggcttaaatctttttttcacctgaagcagtgaccaaggtgtattctgagggaagcttgagttaggtgccttctttaaaacagaaagtcatggaagcacccttctcaagggaaaaccagacgcccgctctgcggtcatttacctctttcctctctccctctcttgccctcgcggtttctgatcgggacagagtgacccccgtggagcttctccgagcccgtgctgaggaccctcttgcaaagggctccacagacccccgccctggagagaggagtctgagcctggcttaataacaaactgggatgtggctgggggcggacagcgacggcgggattcaaagacttaattccatgagtaaattcaacctttccacatccgaatggatttggattttatcttaatattttcttaaatttcatcaaataacattcaggagtgcagaaatccaaaggcgtaaaacaggaactgagctatgtttgccaaggtccaaggacttaataaccatgttcagagggatttttcgccctaagtactttttattggttttcataaggtggcttagggtgcaagggaaagtacacgaggagaggactgggcggcagggctatgagcacggcaaggccaccggggagagagtccccggcctgggaggctgacagcaggaccactgaccgtcctccctgggagctgccacattgggcaacgcgaaggcggccacgctgcgtgtgactcaggaccccataccggcttcctgggcccacccacactaacccaggaagtcacggagctctgaacccgtggaaacgaacatgacccttgcctgcctgcttccctgggtgggtcaagggtaatgaagtggtgtgcaggaaatggccatgtaaattacacgactctgctgatggggaccgttccttccatcattattcatcttcacccccaaggactgaatgattccagcaacttcttcgggtgtgacaagccatgacaacactcagtacaaacaccactcttttactaggcccacagagcacggcccacacccctgatatattaagagtccaggagagatgaggctgctttcagccaccaggctggggtgacaacagcggctgaacagtctgttcctctagactagtagaccctggcaggcactcccccagattctagggcctggttgctgcttcccgagggcgccatctgccctggagactcagcctggggtgccacactgaggccagccctgtctccacaccctccgcctccaggcctcagcttctccagcagcttcctaaaccctgggtgggccgtgttccagcgctactgtctcacctgtcccactgtgtcttgtctcagcgacgtagctcgcacggttcctcctcacatggggtgtctgtctccttccccaacactcacatgcgttgaagggaggagattctgcgcctcccagactggctcctctgagcctgaacctggctcgtggcccccgatgcaggttcctggcgtccggctgcacgctgacctccatttccaggcgctccccgtctcctgtcatctgccggggcctgccggtgtgttcttctgtttctgtgctcctttccacgtccagctgcgtgtgtctctgtccgctagggtctcggggtttttataggcataggacgggggcgtggtgggccagggcgctcttgggaaatgcaacatttgggtgtgaaagtaggagtgcctgtcctcacctaggtccacgggcacaggcctggggatggagcccccgccagggacccgcccttctctgcccagcacttttctgcccccctccctctggaacacagagtggcagtttccacaagcactaagcatcctcttcccaaaagacccagcattggcacccctggacatttgccccacagccctgggaattcacgtgactacgcacatcatgtacacactcccgtccacgaccgacccccgctgttttattttaatagctacaaagcagggaaatccctgctaaaatgtcctttaacaaactggttaaacaaacgggtccatccgcacggtggacagttcctcacagtgaagaggaacatgccgtttataaagcctgcaggcatctcaagggaattacgctgagtcaaaactgccacctccatgggatacgtacgcaacatgctcaaaaagaaagaatttcaccccatggcaggggagtggttggggggttaaggacggtgggggcagcagctgggggctactgcacgcaccttttactaaagccagtttcctggttctgatggtattggctcagttatgggagactaaccataggggagtggggatgggggaacccggaggctgtgccatctttgccatgcccgagtgtcctgggcaggataatgctctagagatgcccacgtcctgattcccccaaacctgtggacagaacccgcccggccccagggcctttgcaggtgtgatctccgtgaggaccctgaggtctgggatccttcgggactacctgcaggcccgaaaagtaatccaggggttctgggaagaggcgggcaggagggtcagaggggggcagcctcaggacgatggaggcagtcagtctgaggctgaaaagggagggagggcctcgagcccaggcctgcaagcgcctccagaagctggaaaaagcggggaagggaccctccacggagcctgcagcaggaaggcacggctggcccttagcccaccagggcccatcgtggacctccggcctccgtgccataggagggcactcgcgctgcccttctagcatgaagtgtgtggggatttgcagaagcaacaggaaacccatgcactgtgaatctaggattatttcaaaacaaaggtttacagaaacatccaaggacagggctgaagtgcctccgggcaagggcagggcaggcacgagtgattttatttagctattttattttatttacttactttctgagacagagttatgctcttgttgcccaggctggagtgcagcggcatgatcttggctcactgcaacctccgtctcctgggttcaagcaattctcgtgcctcagcctcccaagtagctgggatttcaggcgtgcaccaccacacccggctaattttgtatttttagtagagatgggctttcaccatgttggtcaggctgatctcaaaatcctgacctcaggtgatccgcccacctcagcctcccaaagtgctgggattacaggcatgagccactgcacctggcctatttaaccattttaaaacttccctgggctcaagtcacacccactggtaaggagttcatggagttcaatttcccctttactcaggagttaccctcctttgatattttctgtaattcttcgtagactggggatacaccgtctcttgacatattcacagtttctgtgaccacctgttatcccatgggacccactgcaggggcagctgggaggctgcaggcttcaggtcccagtggggttgccatctgccagtagaaacctgatgtagaatcagggcgcgagtgtggacactgtcctgaatctcaatgtctcagtgtgtgctgaaacatgtagaaattaaagtccatccctcctactctactgggattgagccccttccctatccccccccaggggcagaggagttcctctcactcctgtggaggaaggaatgatactttgttatttttcactgctggtactgaatccactgtttcatttgttggtttgtttgttttgttttgagaggcggtttcactcttgttgctcaggctggagggagtgcaatggcgcgatcttggcttactgcagcctctgcctcccaggttcaagtgattctcctgcttccgcctcccatttggctgggattacaggcacccgccaccatgcccagctaattttttgtatttttagtagagacgggggtgggggtggggttcaccatgttggccaggctggtctcgaacttctgacctcagatgatccacctgcctctgcctcctaaagtgctgggattacaggtgtgagccaccatgcccagctcagaatttactctgtttagaaacatctgggtctgaggtaggaagctcaccccactcaagtgttgtggtgttttaagccaatgatagaatttttttattgttgttagaacactcttgatgttttacactgtgatgactaagacatcatcagcttttcaaagacacactaactgcacccataatactggggtgtcttctgggtatcagcgatcttcattgaatgccgggaggcgtttcctcgccatgcacatggtgttaattactccagcataatcttctgcttccatttcttctcttccctcttttaaaattgtgttttctatgttggcttctctgcagagaaccagtgtaagctacaacttaacttttgttggaacaaattttccaaaccgcccctttgccctagtggcagagacaattcacaaacacagccctttaaaaaggcttagggatcactaaggggatttctagaagagcgacccgtaatcctaagtatttacaagacgaggctaacctccagcgagcgtgacagcccagggagggtgcgaggcctgttcaaatgctagctccataaataaagcaatttcctccggcagtttctgaaagtaggaaaggttacatttaaggttgcgtttgttagcatttcagtgtttgccgacctcagctacagcatccctgcaaggcctcgggagacccagaagtttctcgccccttagatccaaacttgagcaacccggagtctggattcctgggaagtcctcagctgtcctgcggttgtgccggggccccaggtctggaggggaccagtggccgtgtggcttctactgctgggctggaagtcgggcctcctagctctgcagtccgaggcttggagccaggtgcctggaccccgaggctgccctccaccctgtgcgggcgggatgtgaccagatgttggcctcatctgccagacagagtgccggggcccagggtcaaggccgttgtggctggtgtgaggcgcccggtgcgcggccagcaggagcgcctggctccatttcccaccctttctcgacgggaccgccccggtgggtgattaacagatttggggtggtttgctcatggtggggacccctcgccgcctgagaacctgcaaagagaaatgacgggcctgtgtcaaggagcccaagtcgcggggaagtgttgcagggaggcactccgggaggtcccgcgtgcccgtccagggagcaatgcgtcctcgggttcgtccccagccgcgtctacgcgcctccgtcctccccttcacgtccggcattcgtggtgcccggagcccgacgccccgcgtccggacctggaggcagccctgggtctccggatcaggccagcggccaaagggtcgccgcacgcacctgttcccagggcctccacatcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgggcctccccggggtcggcgtccggctggggttgagggcggccggggggaaccagcgacatgcggagagcagcgcaggcgactcagggcgcttcccccgcaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggtgaggaggtggtggccgtcgagggcccaggccccagagctgaatgcagtaggggctcagaaaagggggcaggcagagccctggtcctcctgtctccatcgtcacgtgggcacacgtggcttttcgctcaggacgtcgagtggacacggtgatcgagtcgactccctttagtgagggttaattgagctcgcggccgc

SEQ ID NO: 6 - DNA, 7498 bp, mouse
aagcttccagcaaaccagttagagctgagttgatgctctgaagaagagaaaatgtagagacggtactgaacaaataatgtctgggcaaacctcagacatgaaaatggaagacgtggaaatccagagaactctgagggaaaataaaacacaactccaggtcatcacgggactcatcaaactgctgaggtgcagccacagagaaaaatcttaaaatagcctagaacgatgcatgacacataaagcacagagaagacgaagctgagtctgtcttgtaggaacaacttgagaagacctaaaccactgcaatgagtgcattctgctaacttagaatttgctacccagttcagatccaaaaagggtttcacaaagttcaacacaaaacagtagcaggagtggctaagggggacacactgataggaattcagagaagtagggaatgctcatatggggacattacaaaatgtactttcatgttgcttaaatcattttaattgtcaaccacatcaagctaaataatgctttgaggttcataacatttggagattatgtctacactagcagagaaggcaccaataacatcccaattgctagattctcatagaatcatgagtcacaatggcagagacaggttctgagagtgtgtccttgttgtaaacagtatgctctacaaactaagttggctgcaatatcactaggcagtgttgtcccataagacaactatcacatatgtggtccagtgatgaccaaagcatcttttagcattttgcaaatgaagctcaaatcgaatatgactaagctcatgcagtacaaatcaaaggtacactgggatagtttaaaagatacatacttgtactggttagttttgtgtcagcttgacacagctggagttatcacagagaaaagagcttcagttgaggaaattcctccatgagatccagctatagggcattttctcaattagtgatcaaggggggaaggccccttgtgggtgggaccatctctgggctggtagtcttggttctataagagagcaggctgagcaagccaggagaagcaagccagtaaagaacatccctccatggcttctgcatcagctcctgctccctgacctgcttgagttccagttctaacttctttcagtgatgaacagcaatgtggaaatgaaagctgaataaaccctttcctccccattttgcttcttggtcatgatgtttgtgcaggaatagaaaccctgactaagacaatactataaaccctaaaagttgtaaaccaaacacatgtgtttccattaagccatcgtagaacaataagtactcaaccccaagtcacataactataatcccagcctttgaaaaccgggatcaggaattcaaggctagcctcatctatatgtaagattaaagcctgtttgggctgcatgagactttgtttcaaaaaaaaaaaaaaaaaagcaaacaggcaaaaacaaacacaagacaagacagatgtaaaatgaaggaggggtagatgggtcaagtagaaaatagcataggaaacgagtcaagtatagaagaggtggtagtaaccagatcatgcagaaggactcaaggccatctcctcacagtggcttaggtaggccttcctctgctcttgagcaggggcagagttgccgctttaaggaggggatcagtcacctttaagaactgaaaagctgaacagtcttctcaagtcagaagccagtggcttcatcttacacctctcttccttcccttgctactcatattggatctgatgatttgcccaacttggaagaaacatctcttctgaagggtttcacagacaccccatctttccgagaaaggaccgcataggctggccatccctgtgcttacaaaaggaataattaagaaacttaattccataagcaaatacaacctttccaagccccaagtggatgattttatcttactgtttttttatatctcatcaaataacttccaagggctcaaaaatccaaagatgtaaaaaaggaactgagctctgtttgccaagccatgaggattaaataatgacattcaaagagatttttgtgccctaagtactttttattggttttcatagatggtttaatgtgcaagatgaagcaaacagagatgggagtggtatcagcatggattaaggtggcagttgtgagggaggggtactgagagaacaggacaaggtaacctatctaaggagaggccaagttggcaagtgccagggacttctaagcccagaactagtacacattccttaggtgctgtttgggaagtcagggagtcaccagccttgggatctataaaagtgcatggtggcattcactcacatacttcctgagctgttcgatgttgatgaagtcgtgggtatgagactgttgtgtcagtgacaaactatgtaaatgagaatgattgtttccatcttgaccactaagacgtaaaccggttccagtgatctccaaacatggcaagctacagcagagcagcagccccatccagagccttgccctggttctgaatgggggagaatccagtgggagtcggttgctgccagcatgttggggtagaaggctggagcatgacaggtccccgaggatttcctgcttcctatatgggtagggatacttgaggtcctctcttctacctccttccctgcagggtttataacctctaccactgtctgtctctgggatagctcctagggtgcagcccctccccaaaaaggcctctccctggcctcatgtctctaagaacagctttctaaagcaggcctgttacacaaaggctcccttttcctggcttcatcgttgctggtagacaacttccactcgttttccacttcagtttcttctactctgttgttatttgattctgatgcttgaacccagggttgtgtagtcagcaagtgctaccccctccctcctcttctttgtttttttgaggcagggtctcattttgcccaagtggacctaaatttcagcatgtagctggcctggttttgaatgccttctcatcctgcctctacttcccaagagtagcttacaagtgtgcaccaccatgccccgcgatattcttatttttgagactgttttctatgctggtttctttggggaactacactaaggtagcttacaagtgtgcaccaccatgccccgcgatattcttatttttgagactgttttctatgctggtttctttggggaactacactaaggtagcttcattgttggcataaatttctcagttcaggcccatatctcctaagtagcagaactaagcaaatctcaaacaaacccctcaaaaagactgatgtccactaaacggacttctaaaatagctcctgtaatcctgagcatttacaaggcggcagacctcctataagggagtaaatatgaaaacgcgcctgttcaaatgctaggtcggtggatagaagcaatttcctcagaaagctgaaggcaccaaaggttatatttgttagcatttcagtgtttgccaaactcagctacagtagagatcacagattccctatttcccagagattcaaaattcagcagcccctctctaactatggctcagagtcgtgtcattacatatgccccaacaacaacccccacccctatcctacccccgcctcacacgtgcaagtactatcacagttgccaacctagcagagctgccatcctaaggtcgaggtcgccgctttggctgtgtgcacaggcaagcgccctcacccaatggccctggccttgctatgggtgcgtgagttgagatgatgctctggactctgaggtgaaggccactggaacagtgaaaaaagctaacgcagggcttttacctagtccccttcctttggtggtgggtgtttacggaacatatttgggatctgagtgtatggtcgcaccacaataaagccttaacctatatagtagaatttcagctgtaatcattaagaactgagattgccaccacccacctcactgtctgtgtcaaccacagcaggctggagcagtcagctcaggaacaggcaaaaccttaggtccctccgcctacctaaccttcaatacatcaaggataggcttctttgcttgcccaaacctcgccccagtctagaccacctggggattcccagctcagggcgaaaaggaagcccgagaagcattctgtagagggaaatcctgcatgagtgcgccccctttcgttactccaacacatccagcaaccactgaacttggccggggaacacacctggtcctcatgcaccagcattgtgaccatcaacggaaaagtactattgctgcgaccccgccccttccgctacaacgcttggtccgcctgaatcccgccccttcctccgttcccagcctcatctttttcgtcgtggactctcagtggcctgggtcctggctgttttctaagcacacccttgcatcttggttcccgcacgtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgggcctccaggcgggatccccatgggtcaggggcggaaagccgggaggacgtgggatagtgcgtctagctcatgtgtcaagaccctcttctccttaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccgggtgagcatggctggtctccagctgaatgcattaggggcccagaaaagggagacaatgggtggcagtaacccaggtccccagtggtgtggtggctttatgcagtccgtggttggatgagttccatcttatggtctctgactccaagctccctccagctcgccttgcacaaactaagattcttgtccaagccctgggcaggttctcagggctggggacattgtggtgaacagataagcagacggggagcatggtggataggagttctggcacagtgcaccagagagagtctggaagcgctagtgagagctaatgtaagggcccgtggttcgccaaagaatgataaccccggactcaaatagtatgccaaagcaaggagcatttcattctgcagaaatcaagcatgcaggtggggggggggggttgctctcattccaagatggagagacaaccaagtatagattttaaggggatcgggggcctttatcttactccatctctaggggcattccattactggggcatggggttggaggttggaaactgttaatggggaggtctggaaacttgctgccccattgtccttgcttcaggctaggtagctgagtagcttctaatggcaggatagtttctgactagctgtctaaagtctggggtgtttgtttttttgttttttctagtaacttacttgcctgaacttgctcagtttttaggcctggtctcctggactgccaatttgaagcctattaaggagtcagcctgtctcactactccaggttatctataatccccctgtagaacggtacctcactgataacaatgacagaccaacataggaacccactatccttgtggtgcatgagtttcaaaggttcttctggtcctcccagtgtgcagatccatgcttaagctatggtcctcccagtgtgcagatccgtgcttaagctatggtcttgcagctgctcgatctacaaagggtagggtgaacgaaggaaagataaatgaaaaaaaaaaaactgtttcctacagtgaagatcgctgccccatcttagctatgagaagggactggggagtggagcctggtgcataaaagaggattgtgttacttggaaggctgcagagcctggactcctgtgccctccttgcctggttttctgggtttaatgttgaggttggccctctgtagtcactacctgaccccttccctttcagccaaccctccggttacaccctgtgcatgtatggaaggggccaaacgccctatcctgctctcccttccccaaaattcttaggatattaacaacttatggggaaaagatggtagagctatgtttacccaccatgtacttgggaagctccgaagtaagctt

SEQ ID NO: 7 - DNA, 261 bp, human
ccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatg

SEQ ID NO: 8 - DNA, 68 bp, human
ccctccccttcctttccgcggccccgccctctcctcgcggcgcgagtttcaggcagcgctgcgtcctg

SEQ ID NO: 9 - DNA, 59 bp, human
ccctccccttcctttccgcggccccgccctctcctcgcggcgcgagtttcaggcagcgc

SEQ ID NO: 10 - DNA, 58 bp, human
ccctccccttcctttccgcggccccgccctctcctcgcggcgcgagtttcaggcagcg

SEQ ID NO: 11 - DNA, 57 bp, human
ccctccccttcctttccgcggccccgccctctcctcgcggcgcgagtttcaggcagc

SEQ ID NO: 12 - DNA, 67 bp, human
cctccccttcctttccgcggccccgccctctcctcgcggcgcgagtttcaggcagcgctgcgtcctg

SEQ ID NO: 13 - DNA, 58 bp, human
cctccccttcctttccgcggccccgccctctcctcgcggcgcgagtttcaggcagcgc

SEQ ID NO: 14 - DNA, 57 bp, human
cctccccttcctttccgcggccccgccctctcctcgcggcgcgagtttcaggcagcg

SEQ ID NO: 15 - DNA, 57 bp, human; identical to SEQ ID NO: 11